(19) United States
(12) Patent Application Publication
(10) Pub. No.: US 2021/0353692 A1
(43) Pub. Date: Nov. 18, 2021

(54) METHOD FOR PREVENTING OR TREATING ABNORMAL EMOTION OR BEHAVIOR IN NON-HUMAN ANIMAL BY LACTIC ACID BACTERIUM

(71) Applicant: BENED BIOMEDICAL CO., LTD., Taipei City (TW)

(72) Inventors: Ying-Chieh TSAI, Taipei City (TW); Lih-Chiann WANG, Taipei City (TW); Chin-Lin HUANG, Taipei City (TW); Chien-Chen WU, Taipei City (TW); Chih-Chieh HSU, Taipei City (TW)

(73) Assignee: BENED BIOMEDICAL CO., LTD., Taipei City (TW)

(21) Appl. No.: 17/286,248

(22) PCT Filed: Jan. 22, 2020
(86) PCT No.: PCT/CN2020/073755

§ 371 Date: Apr. 16, 2021

Related U.S. Application Data
(60) Provisional application No. 62/800,150, filed on Feb. 1, 2019.

(51) Int. Cl. A61K 35/747; A61P 25/00

(52) U.S. Cl. CPC A61K 35/747; A61P 25/00

(57) ABSTRACT

Provided is a method for preventing or treating an emotional or behavioral problem in a non-human animal, such as fears, phobias, anxieties and aggression, by administering a lactic acid bacterium the non-human animal in need thereof

### BACKGROUND

### 1. Technical Field

This disclosure relates to methods for preventing or treating emotional and behavioral problems in animals, and relates particularly to methods for preventing or treating fears, phobias, anxieties and aggression in non-human animals.

### 2. Description of Related Art

The health benefits of companion or domestic animals have been widely recognized in the public. Research shows that pet owners have less illness, recover faster from serious health conditions, and tend to be more content than people who do not own pets. Some specific potential benefits of owning companion animals include fewer feelings of loneliness, more compatibility among humans, fewer allergies, lower blood pressure, and enhanced ability to meet others and be more social, which are strongly associated with good mental and physical health.

However, just like human and other animals, companion and domestic animals can develop emotional and behavioral problems as well. These problems could weaken the pet-owner bond, resulting in a decreased owner commitment to pet care. In fact, the emotional and behavioral problems are a primary reason for pet relinquishment to a humane society or shelter, abandonment, or euthanasia. For example, one frequently cited reason for returning a dog is behavior attributed to separation anxiety. It has been estimated that in the average veterinary practice in the United States, up to 14% of canine patients exhibit one or more signs of separation anxiety, including abnormal elimination, destruction, and vocalization. Other behaviors associated with anxiety may include salivation, anorexia, and lethargy.

Thus, when these problems develop in companion and domestic animals, they require immediate attention and treatment. In companion animals, the treatment of behavioral problems varies with diagnosis and prognosis. In general, the treatment program begins with behavior modification that starts with prevention and avoidance of problems. Initially, prevention is necessary to avoid further compromising the pet's welfare and to ensure safety in cases of aggression. At the same time, the owner needs to develop effective strategies to modify the pet's behavior so that it might gradually be reintroduced to the problem situations while achieving a desirable outcome. Repetition of the behavior further aggravates the problem if the pet successfully accomplishes its intended goal (e.g., escape or retreat from the stimulus), while each exposure in which the outcome is unpleasant can condition further anxiety. Improvement is generally a slow and gradual process, and modifications to the environment may be required, so that the pet can be kept away from the stimuli (or the sights or sounds of the stimuli). Modifying the pet's behavior is accomplished by applying the principles of learning and behavior modification, primarily achieving and rewarding desirable outcomes along with use of products that improve safety, reduce anxiety, or help to achieve the desired response more effectively (e.g., muzzles, head halters, no-pull harnesses, etc.).

Drugs and natural products may also be indicated for some pets and some problems. A variety of natural products have been used to treat anxiety; however, only a few have demonstrated evidence to support efficacy. There has been recommendation on the psychoactive drugs as adjuncts to behavior modification in the treatment of selected cases of nuisance barking, such as amitriptyline, buspirone, clomipramine, and fluoxetine.

Accordingly, there remains an unmet need to provide an effective prevention or treatment for abnormal emotions and behaviors in non-human animals.

### SUMMARY

In this disclosure, it is found that a lactic acid bacterium, Lactobacillus plantarum subsp. plantarum PS128 (hereinafter referred to as PS128), is effective in preventing or treating an abnormal emotion or behavior in a non-human animal, e.g., for preventing or treating fears, phobias, anxieties and aggression.

In one embodiment of the present disclosure, a method for preventing or treating an abnormal emotion or behavior in a non-human animal is provided. In one embodiment, the non-human animal is a companion animal, a domestic animal, a pet, a farm animal or a pack animal. In another embodiment, the non-human animal is a dog, a cat, a goat, a pig, a sheep, a cattle, a chicken, a donkey, a llama, a camel, an elephant, a monkey, a chimpanzee, a gorilla, a deer, a duck, a goose, a pigeon, a turkey, a rabbit, a squirrel, a mouse, a rat, a hamster, a guinea pig or a horse. In accordance with the present disclosure, the method comprises administering a composition, wherein the composition comprises an effective amount of a Lactobacillus plantarum subsp. plantarum PS128, which is deposited under DSMZ Accession No. DSM 28632, and a carrier. In one embodiment of the present disclosure, the composition is orally administrated to the non-human animal. In one embodiment, the effective amount of PS128 administered per kilogram bodyweight per day is at least 1×10⁷ CFU, at least 1×10⁸ CFU, at least 1×10⁹ CFU, at least 1×10¹⁰ CFU or at least 1×10¹¹ CFU, including 1×10⁷ CFU, 2×10⁷ CFU, 3×10⁷ CFU, 4×10⁷ CFU, 5×10⁷ CFU, 6×10⁷ CFU, 7×10⁷ CFU, 8×10⁷ CFU, 9×10⁷ CFU, 1×10⁸ CFU, 2×10⁸ CFU, 3×10⁸ CFU, 4×10⁸ CFU, 5×10⁸ CFU, 6×10⁸ CFU, 7×10⁸ CFU, 8×10⁸ CFU, 9×10⁸ CFU, 1×10⁹ CFU, 2×10⁹ CFU, 3×10⁹ CFU, 4×10⁹ CFU, 5×10⁹ CFU, 6×10⁹ CFU, 7×10⁹ CFU, 8×10⁹ CFU, 9×10⁹ CFU, 1×10¹⁰ CFU, 2×10¹⁰ CFU, 3×10¹⁰ CFU, 4×10¹⁰ CFU, 5×10¹⁰ CFU, 6×10¹⁰ CFU, 7×10¹⁰ CFU, 8×10¹⁰ CFU, 9×10¹⁰ CFU, 1×10¹¹ CFU, 2×10¹¹ CFU, 3×10¹¹ CFU, 4×10¹¹ CFU, 5×10¹¹ CFU, 6×10¹¹ CFU, 7×10¹¹ CFU, 8×10¹¹ CFU, and 9×10¹¹ CFU, but not limited thereto.

In one embodiment, the dosage of PS128 administered per kilogram bodyweight per day is at least 0.01 g, at least 0.02 g, at least 0.03 g, at least 0.04 g or at least 0.05 g. In another embodiment, the dosage of PS128 administered per kilogram bodyweight per day is 0.01 g, 0.015 g, 0.02 g, 0.025 g, 0.03 g, 0.035 g, 0.04 g, 0.045 g or 0.05 g.

In one embodiment of the present disclosure, the abnormal emotion or behavior is selected from the group consisting of fear, phobia, anxiety and aggression. In a further embodiment, the abnormal emotion or behavior includes one of vocalization, hyperactivity, destruction, abnormal sleep, abnormal feeding, abnormal drinking, abnormal grooming, abnormal elimination, socialization disorder, compulsive behavior, barking, growling, biting, mouthing, separation anxiety, play aggression, and any combination thereof.

In one embodiment of the present application, a score or a value for evaluating the abnormal emotion or behavior in the non-human animal is improved. In a further embodiment, the evaluating includes evaluation of dog's emotional and cognitive disorders (EDED) or scoring of canine behavior checklist.

In one embodiment of the present disclosure, after the administration of the composition, the value of the evaluation of dog's emotional and cognitive disorders (EDED value) is statistically significantly decreased. In another embodiment, after the administration of the composition, at least a score of canine behavior checklist is statistically significantly decreased. In still another embodiment, after the administration of the composition, the total score of canine behavior checklist is statistically significantly decreased.

### BRIEF DESCRIPTION OF THE DRAWINGS

FIG. 1 shows the total scores of the canine behavior checklist before and after oral administration of PS128 (data shown as the means±S.D., n=20, p<0.001).

FIG. 2 shows the EDED value before and after oral administration of PS128 (data shown as the means±S.D., n=20, p<0.005).

### DETAILED DESCRIPTION OF THE EMBODIMENTS

The following examples are used for illustrating the disclosure. A person skilled in the art can easily conceive the other advantages and effects of the disclosure from these examples. The disclosure can also be implemented by various modifications and changes that do not depart from the spirit of the disclosure. It is understood that the examples provided below are only exemplary of the disclosure and should not be taken as a limit to the scope of the disclosure.

### Fears and Phobias

Fear is a normal response to an actual or perceived threatening stimulus or situation. Apprehension or anxiety is a response to fear and agitation, when the animal anticipates a threat or fearful situation. Phobia is an exaggerated fear response. The fear response may include panting and salivation, tucked tail, lowered ears, gazing away, low body posture, piloerection, vocalization, or displacement behaviors such as yawning or lip licking. While avoidance and escape is one strategy, some dogs use aggression to remove the fear-evoking stimulus and are reinforced by success (negative reinforcement).

Some of the more common presentations include the following: 1) fear of other dogs, especially those that are unfamiliar, appearing threatening to the dog, or with which the dog has had an unpleasant experience; 2) fear of unfamiliar people, especially those who are novel or look, act, or smell different than those the dog is accustomed to (e.g., young children); 3) fear of inanimate stimuli such as loud or unfamiliar noises (e.g., construction work, trucks, gunshot), visual stimuli (e.g., umbrellas, hats, uniforms), environments (e.g., backyard, park, boarding kennel), surfaces (e.g., grass, tile or wood floors, steps), or a combination of stimuli (e.g., vacuum cleaners, car rides); and 4) fear of specific situations such as veterinary clinics or grooming parlors.

Phobic responses in dogs are generally associated with loud noises (e.g., thunder, fireworks, gunshots), and the stimuli associated with these events include rain, lightning, and perhaps even static or pressure changes associated with a thunderstorm. Some fears (e.g., veterinary clinics, going outdoors, entering certain rooms, or walking on certain types of flooring) may become so intense that they meet the definition of a phobia.

### Separation Anxiety

Separation anxiety is an inability of the pet to find comfort when separated from family members. The problem may be primary (e.g., hyperattachment, dysfunctional attachment) as the puppy ages and matures; in fact, the chances of the problem developing can be reduced by having puppies regularly spend time during the day on their own (e.g., in a safe haven). In other cases, the anxiety about being left alone is secondary to an event such as a change in the household or dog's daily routine, or associated with an underlying state of anxiety along with other behavioral issues such as noise phobias and separation anxiety. Anxiety may lead to destructive behavior (e.g., at exits or toward owner possessions), distress vocalization, house soiling, salivation, pacing, restlessness, inability to settle, anorexia, and repetitive or compulsive behaviors. The behaviors are exhibited when the dog is left alone, and generally arise within the first 15 to 30 min after departure. Many pets with separation anxiety begin to exhibit signs as the owner prepares to depart (e.g., putting on shoes, getting keys, or going to the door). When the owner is home, the dog may crave constant contact or proximity to the owner. When the owner returns, the welcoming responses are commonly exaggerated, and the dog is hard to calm down.

### Aggression

Aggression refers to threatening behaviors or harmful attacks and can range from subtle changes in body posture, facial expressions, and vocalization to biting, and can be fear-related aggression, possessive aggression (resource guarding), play aggression, redirected aggression, irritable/conflict/impulse control aggression, aggression toward other dogs, territorial/protective aggression, and predatory aggression.

### EXAMPLES

### Example 1

### Preparation of PS128

Lactobacillus plantarum subsp. plantarum PS128 (hereinafter referred to as PS128) used in the study was the strain isolated and deposited under DSMZ Accession No. DSM 28632. PS128 was inoculated in a culture medium (10% skim milk, 1% yeast powder, 0.1% Tween 80, and 2% glucose), cultured at 37° C. for 18 hours and harvested by centrifugation. PS128 was embedded and lyophilized with protective agents (1% skim milk, 2% sugar, 2% oligofructose, 3% maltodextrin, and 2% glycerol) and excipients to a final concentration of 5×10¹⁰ colony formation unit (CFU) per gram powder.

### Example 2

### Treating Abnormal Emotions and Behaviors with PS1285

A total of 20 dogs were examined in the veterinary clinic for their abnormal emotions and behaviors. They were evaluated and given score by professional veterinarian with the canine behavior checklist as shown in Table 1 and the Evaluation of Dog's Emotional Disorders (EDED) scale as shown in Table 2 below. The checklist and the EDED scale are used by the veterinarian for evaluating abnormal emotions and behaviors in dogs (Landsberg G, Hunthausen W, Ackerman L., 2013, Behavior Problems of the Dog and Cat. Saunders, Edinburgh 2013, Elsevier Ltd.). Total scores were calculated and obtained for each dog. The tested dogs were further examined by veterinarian to exclude the abnormal behavior due to illness, such as the abnormal behavior due to abnormal thyroxine.

TABLE 1
Canine behavior checklist
Canine behavior checklist
Name: Today's date:

| | |
|---|---|
| Pet's name: Age: Sex: M/F | Neutered: Y/N |

Please use the following scoring system:
Scoring: 0 - never; 1 - rarely; 2 - sometimes; 3 - frequent; 4 - all the time

| | | When |
|---|---|---|
| | Score | began? |
| 1. Fear (no aggression): People/Locations/Situations/Noises | | |
| People familiar unfamiliar | | |
| Animals familiar unfamiliar | | |
| Car rides Veterinary clinic Surfaces Storms | | |
| Fireworks Vacuum Other: | | |
| Describe: | | |
| 2. Growl/threaten/bite unfamiliar people | | |
| Describe: | | |
| 3. Growl/threaten/bite family members | | |
| Describe: | | |
| 4. Growl/threaten/bite other family pets | | |
| Describe: | | |
| 5. Growl/threaten/bite unfamiliar dogs | | |
| Describe: | | |
| 6. Mouthing/grabbing/play biting | | |
| Describe: | | |
| 7. When left alone: | | |
| anxious destructive vocal soils salivate | | |
| 8. Soiling indoors: urine stools | | |
| Left alone: Family at home | | |
| Describe: | | |
| 9. Destructive: Chews Digs Other: | | |
| 10. Steals: garbage food toys Other: | | |
| Describe: | | |
| If yes, will he drop/give? Y N | | |
| Is he possessive/aggressive? Y N | | |
| 11. Barking at doors, windows, fences? Other: | | |
| Describe: | | |
| 12. Excitable/won't settle Jumps on people | | |
| Gets on fumiture /counters | | |
| Describe: | | |
| 13. Difficult to train Ignores commands Pulls on walks | | |
| Other: | | |
| Describe: | | |
| 14. Repetitive behaviors - | | |
| Chews/licks self Chases tail Licks/suck Staring | | |
| Circling Snaps at air Chases lights Other: | | |
| Describe: | | |
| 15. Mounting other dogs Household objects Masturbate | | |
| 16. Chases people animals cars bikes wildlife | | |
| Other: | | |
| Describe: | | |
| 17. Ingestive - Eating: | | |
| Voracious/gulps Picky Excessive drinking | | |
| Eats stool Eats other nonfood items (pica) | | |
| Describe: | | |
| 18. Attention seek Clingy | | |
| Describe: | | |
| 19. Wakes at night | | |
| 20. Other/describe: | | |
| If you need assistance with any of the above - indicate # | | |

TABLE 2
Evaluation of dog's emotional and cognitive disorders (EDED scale)
The EDED scale - evaluation of dog's emotional and cognitive disorders

| | | | | |
|---|---|---|---|---|
| Behavior type | Specific behavior | Score | Date | Score |

Centripetal

| | | |
|---|---|---|
| Feeding | Normal appetite | 1 |
| | Hyperphagia (with regurgitation | 3 |
| | and reingestion)¹ | |
| | Anorexia/hyporexia | 4 |
| | Dysorexia (moving from hyper to | 5 |
| | hypo) | |
| Drinking | Normal drinking | 1 |
| | Carries empty water bowl around | 2 |
| | (ritual)² | |
| | Chews at water without | 3 |
| | swallowing³ | |
| | High-frequency drinking | 5 |
| | (documented) | |
| Self-stimulatory | Normal cleaning behavior | 1 |
| | Excessive licking, nibbling⁴ | 4 |
| | Stereotyped nibbling, dizziness, | 5 |
| | turning on itself (or other | |
| | stereotypies)⁵ | |
| Sleep | Normal (or no change) | 1 |
| | Increase in sleep, hypersomnia⁶ | 2 |
| | Insomnia, during sleep (and | 3 |
| | hyposomnia)⁷ | |
| | Wakes up shortly after falling asleep, | 5 |
| | anxiety at time of going to sleep (and | |
| | restlessness)⁸ | |

Centrifugal

| | | |
|---|---|---|
| Exploratory | Normal | 1 |
| (scanning) | Inhibited | 2 |
| | Frequent avoidance responses | 3 |
| | Increased, hypervigilant | 4 |
| | Oral | 5 |
| Aggression | No aggression or aggression stable | 1 |
| (defense) | (no increase or decrease) | |
| | Irritation-related aggression | 3 |
| | Fear-related aggression | 4 |
| | Displays both fear and irritation | 5 |
| | aggression | |
| Learned social | Unchanged | 1 |
| behavior | No submission response | 2 |
| | No self-control when playing | 2 |
| | Bites without growling | 4 |
| | Steals, does not drop stolen objects | 5 |
| Specific learned | Same response capacity (allowing for | 1 |
| behavior | disease or age) | |
| | Arbitrary responses | 3 |
| | No response to previously learned | 5 |
| | behaviors | |
| Physical exam⁹ | Normal | 1 |
| | Periods of tachycardia and/or | 2 |
| | tachypnea | |
| | Diarrhea, colic | 2 |
| | Dyspepsia (and ptyalism) | 2 |
| | Increased emotional micturition | 3 |
| | Acral lick granuloma (and extensive | 4 |
| | lick alopecia) | |
| | Obesity | 4 |
| | High-quantity drinking and urination | 4 |
| | (PU/PD) | |
| Total | | |

Interpretation grid of EDED scores

| EDED value | Interpretation |
|---|---|
| 9-12 | Normal state |
| 13-16 | Phobias |
| 17-35 | Anxieties |
| 36-44 | Emotional (thymic) disorders |

Centripetal, internal factors; centrifugal, external stimuli; PC, polyuria; PD, polydipsia.
¹Hyperphagia with regurgitation and reingestion. The dog eats rapidly, displays spasms, followed by vomiting. It then reingests what it has just expelled and resumes its meal. This behavior appears regularly (1 meal in 2).
²Carries empty water bowl around. The dog moves or carries its bowl toward one or several family members. This behavior stops as soon as the bowl is filled.
³Chews at water without swallowing it (dipsomania). The dog nibbles the water and spreads it around its bowl while swallowing very little.
⁴Excessive licking, nibbling. A dog that is licking or nibbling itself, then spontaneously stops.
⁵Stereotypic nibbling, dizziness. When there is licking or nibbling that does not stop spontaneously (the owners must stop the dog or divert its attention) or else dizziness or any other stereotypy (e.g., licking of the face, jumping, wandering).
⁶Increase in sleep, hypersomnia. When the duration of sleep is longer than the age norm (+25%).
⁷Insomnia, during sleep. Awakenings appear more than 90 minutes after going to sleep.
⁸Wakes up shortly after going to sleep, anxiety at time of going to sleep. The dog awakes in the 30-45 minutes following going to sleep. Prior to going to sleep the dog may display moans, excitement, and a search for contacts, as if it is afraid of going to sleep.
⁹In order to take the physical examination into account, all the manifestations observed must be scored and counted.

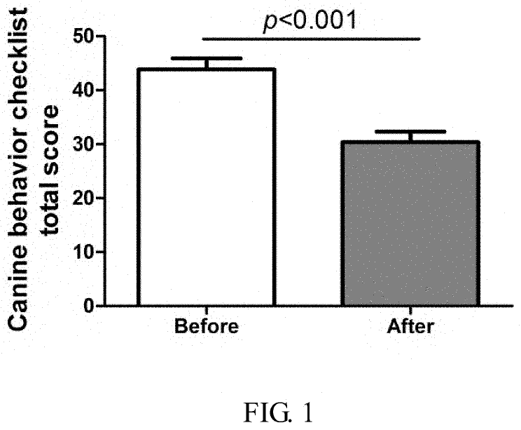

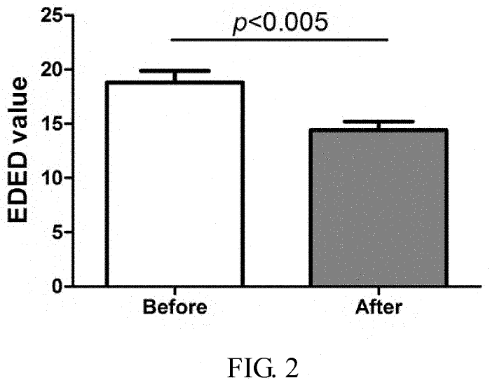

After the dogs were evaluated in the veterinary clinic for their abnormal emotions and behaviors, they were given PS128 at a dosage of 0.02 g/kg bodyweight/day for a period of 2 weeks. After administration of PS128 for 2 weeks, they were evaluated again for their abnormal emotions and behaviors with the same behavior checklist and EDED scale. The results of the behavior checklist were summarized in FIG. 1, and the results of the EDED scale were illustrated in FIG. 2. The total score of the evaluation before and after administration of PS128 were recorded and analyzed.

As shown in FIGS. 1 and 2, the total scores of both the canine behavior checklist and the EDED scale decreased significantly after PS128 administration compared to those before the PS128 administration, indicating the effect of PS128 in preventing and treating abnormal emotions and behaviors in these companion animals.

While some of the embodiments of the present disclosure have been described in detail in the above, it is, however, possible for those of ordinary skill in the art to make various modifications and changes to the embodiments shown without substantially departing from the teaching and advantages of the disclosure. Such modifications and changes are encompassed in the scope of the disclosure as set forth in the appended claims.

What is claimed is:

1. A method of preventing and treating an abnormal emotion or behavior in a non-human animal comprising administering to the non-human animal in need thereof a composition comprising an effective amount of Lactobacillus plantarum subsp. plantarum PS128, which is deposited under DSMZ Accession No. DSM 28632, and a carrier thereof.

2. The method of claim 1, wherein the abnormal emotion or behavior is selected from the group consisting of fear, phobia, anxiety, aggression, vocalization, hyperactivity, destruction, abnormal sleep, abnormal feeding, abnormal drinking, abnormal grooming, abnormal elimination, socialization disorder, compulsive behavior, barking, growling, biting, mouthing, separation anxiety, play aggression, and any combination thereof

3. The method of claim 1, wherein the non-human animal is a companion animal, a domestic animal, a pet, a farm animal or a pack animal.

4. The method of claim 1, wherein the non-human animal is selected from the group consisting of a dog, a cat, a goat, a pig, a sheep, a cattle, a chicken, a donkey, a llama, a camel, an elephant, a monkey, a chimpanzee, a gorilla, a deer, a duck, a goose, a pigeon, a turkey, a rabbit, a squirrel, a mouse, a rat, a hamster, a guinea pig and a horse.

5. The method of claim 1, wherein the Lactobacillus plantarum subsp. plantarum PS128 is orally administrated to the non-human animal in an amount of at least 10⁷ CFU per day.

6. The method of claim 1, wherein the Lactobacillus plantarum subsp. plantarum PS128 is orally administered to the non-human animal at a dosage of at least 0.01 g/kg bodyweight/day.

7. The method of claim 1, wherein the Lactobacillus plantarum subsp. plantarum PS128 is administered to the non-human for at least one week.

8. The method of claim 1, further comprising improving a score or a value for evaluating the abnormal emotion or behavior in the non-human animal.

9. The method of claim 8, wherein the evaluating includes evaluation of dog's emotional and cognitive disorders (EDED), and an EDED value in the non-human animal is decreased.

10. The method of claim 8, wherein the evaluating includes scoring of canine behavior checklist, and at least a score of the canine behavior checklist in the non-human animal is decreased.
